(19) United States
(12) Patent Application Publication
(10) Pub. No.: US 2005/0153933 A1
(43) Pub. Date: Jul. 14, 2005

(54) Use of compounds comprising a polysaccharide structure as biofertiliser and phytosanitary products

(76) Inventors: Yvette Janine Lienart, Uriage (FR); Alain Charles Abel Heyraud, Voroize (FR)

Correspondence Address: YOUNG & THOMPSON, 745 SOUTH 23RD STREET, 2ND FLOOR, ARLINGTON, VA, 22202, US

(21) Appl. No.: 10/509,070

(22) PCT Filed: Mar. 27, 2003
(86) PCT No.: PCT/FR03/00969

(30) Foreign Application Priority Data
Mar. 27, 2002 (FR) 02/03849

(51) Int. Cl. A61K 31/715; A01N 43/04

(52) U.S. Cl. 514/61; 504/117

(57) ABSTRACT

The invention relates to the use of a compound comprising a polysaccharaide structure having formula XFG, or a derivative structure, in relation to adapting plants to abiotic stress, controlling flowering and fructification and inducing defence reactions against pathogens.

A subject of the present invention is new uses of compounds comprising an osidic structure containing X, F and G chains, as well as of derived compounds, in the phytosanitary field, and that of biofertilization.

The cell walls of fruits and vegetables are formed by polysaccharides, of which chiefly pectin, cellulose and xyloglucan are involved in putting the walls in place (Levy S et al., Plant J. 1997, 11(3): 373-86). Xyloglucan is also found in large quantities in the endosperm of the seeds of the Dicotyledons.

Xyloglucan is a 1,4-β-glucan polymer substituted differently according to its origin. In the Dicotyledons, the substitutions of the linear 1,4 β-D-glucan chains most often involve 1,6 α-D-xylosyl-, or 1,6 α-D-xylose 1,2 β-D-galactosyl-type branchings, and fucose can be associated, at the terminal position, with the galactose, i.e. a 1,6 α-D-xylose 1,2 β-D-galactose 1,2 α-L-fucosyl-type side branching. Always in the Dicotyledons, the fucose residue is absent from the endosperm, and it can be replaced by the α-L-arabinose residue, for example in certain Solanaceae. The xyloglucan of the Monocotyledons differs from that of the Dicotyledons by a lower rate of substitution by the xylose, galactose residues and by the absence of fucose. The xyloglucan forms with the cellulose microfibres the bridge structures which constitute the structure and ensure the flexibility of the cell wall of vegetables (Pauly M, Albersheim P, Darvill A, York W S (1999) Plant J, 20 (6): 629-39).

Xyloglucan is a substrate of endoxyloglucanases (Vincken J P, Beldman G, Voragen A G Carbohydr Res (1997) 13, 298(4): 299-310) or of xyloglucan endotransglycosylase (Steele N M, Fry S C, Biochem J (1999) 15, 340, 1, 207-211), namely of enzymatic activities capable of modifying the structure of the cell walls during cell elongation, in the germination, fructification periods for example and which are dependent on hormones, in particular auxins (Hetherington P R and Fry S. (1993) Plant Physiology, 103, 987-992), and gibberellins (Maclachlan G and Brady C (1994) Plant Physiol 105, 965-974).

Xyloglucan, in particular a fucosylated oligomer, the nonasaccharide XXFG (described in Fry et al. (1993) Physiologia Plantarum, 89, 1-3), is well known for its antiauxinic effect (Mac Dougall C J and Fry S C (1989) Plant Physiol 89, 883-887). Conversely, oligomers without fucose but with galactose such as the oligomers XXLG and XLLG have an auxinic effect (Mc Dougall G J and Fry S C (1990) Plant Physiology 93, 1042-1048).

Moreover, a number of signals generate activated oxygen species (also referred to as “oxidative burst”). Active oxygen species are well known for being released during plant-pathogen interactions. Oligosaccharides of various origin (polygalacturonic acid, chitosan, O-glycans etc.) have been recorded-for their ability to generate an oxidative burst (Low P S and Heinstein P F (1986) Arch. Biochem. Biophys. 249, 472-479; Rogers K R., Albert F, and Anderson A J (1988) Plant Physiol 86, 547-553; Apostol I, Heinstein P F and Low P S (1989) Plant Physiol 90, 109-116; Vera-Estrella R, Blumwald E and Higgins V J (1992) Plant Physiol. 1208-1215; Bolwell G P, Butt V S, Davies D R and Zimmerlin A. (1995) Free Rad. Res. Comm. 23, 517-532; Orozco-Cardenas M and Ryan C A (1999) PNAS, 25, 96, 11, 6553-655; Nita-Lazar M, Iwahara S, Takegawa K, Lienart Y (2000) J Plant Physiol, 156, 306-311). Oxidoreductase NAD(P)H enzymes for the release of superoxide anion (Van Gestelen P V, Asard A, Caubergs R J (1997) Plant Physiol 115, 543-550) and peroxidase enzymes for the formation of peroxide or of superoxide anion or of OH radicals are involved (Baker C J and Orlandi E W (1995) Ann. Rev. Phytopathol, 33,299-321; Chen S X and Schopfer P (1999) Eur Bioch 260, 726-735). Other signals (salicylic acid, jasmonates, cGMP, NO etc.) also generate a burst (Chen Z, Malamy J, Henning J, Conrath U, Sanchez-Casas P, Silva H, Ricigliano J, Klessig D F (1995) Proc Natl Acad Sci USA, 92, 4134-4137; Voros K, Feussner I, Kuhn H, Lee J, Graner A, Lobler M, Parthier B, Wastemack C Eur J Biochem (1998) 15, 251, 36-44; Durner J, and Klessig J, Wendehenne D, Klessig D F (1998) Proc Natl Acad Sci USA, 95, 10328-10333; Durner D atid Klessig D F (1999) Current Opinion in Plant Biology, 2, 369-374).

Extreme environmental conditions (drought, cold, UW, salinity etc.) trigger the same effect.

The major role of H₂O₂ in the generation of the burst as in the regulation of oxidant stress is based on:
its formation by dismutation from the superoxide anion (Bolwell G P, Davies D R, Gerrish C, Auh C K and Murphy T M (1998) Plant Physiol 116, 1379-1385),
its use in C₁₈ fatty acid metabolism sequences (for the peroxidation of lipids (Koch E, Meier B M, Eiben H-G, Slusarenko A (1992) Plant Physiol 99, 571-576) or for the synthesis of octadecanoids and of their derivatives, certain of which such as the methyl-jasmonates are metabolites with a hormonal function,
its function as substrate for the peroxidase and catalase enzymes, property of limiting the accumulation of toxic peroxide for the cell (Baker C J, Harmon G L, Glazener J A and Orlandi E W (1995) Plant Physiol, 108, 353-359). The active oxygen species, the superoxide anion in particular, control different metabolic routes. They are involved in:
the biosynthesis of polyamines: monoamines are oxidized to aldehydes with production of NH₃ and peroxide. The oxidation of L-arginine by nitrite synthase results in the formation of a polyamine precursor (L-citrulline),
the synthesis of ethylene,
the synthesis of gibberellins. More than 20 oxidases are involved in the regulation of the biosynthesis of gibberellins.

The active oxygen species are involved in signal transduction stages, because they are associated with receptor bond activity or transduction enzyme activity (Jabs T, Tschöpe M, Colling C, Hahlbrock K and Scheel D (1997) Proc Natl Acad Sci USA 29, 94, 9, 4800-4805; Durner J, Wendehenne D, Klessig D F (1998) Proc Natl Acad Sci USA, 95, 10328-10333).

They are involved in the regulation of the cell redox potential using thiol groups (GSSG-GSH, cystine-cysteine conversion, etc.). In this way, they control senescence processes which are manifested during certain flowering and fructification phases in different organisms.

The oxidative burst interferes with the hormonal metabolism, the most efficient potential for regulating the flowering and fructification stages (in particular their triggering and their duration are programmed by a hormonal balance (auxin/cytokinin ratio for example), and the active oxygen species, including peroxide, control the synthesis of polyamines).

The present invention results from the revealing by the Inventors of the fact that the compounds comprising an osidic structure of formula XFG, as well as compounds derived from the latter, have a stimulating effect on the glutathione reductase enzyme, the phospholipase D enzyme in plants, as well as the glycosylhydrolases.

By stimulating the glutathione reductase enzyme, the compounds of the invention trigger the reactions of adaptation to any oxidant stress, such as cold in particular, by limiting the toxic effects of the active oxygen species (Allen R D, Webb R P, Schake I T S (1997) Free Radic Biol Med, 23 (3): 473-479; O'Kane D, Gill V, Boyd P, Burdon R (1996) Planta, 198 (3): 371-377), and they regulate the redox-potential of the cell, which modifies the activity of enzymes or of thiol-dependent proteins, phospholipase D, thiol-proteases and inhibitors of thiol-proteases in particular (Taher M M, Mahgoub M A, Abd-Elfattah (1998) AS Biochem Mol Biol Int 46 3, 619-28), as well as by a thiol-dependent protease inhibitor induction effect, and without however activating a cascade of other enzymatic systems in proportions harmful to the plant.

By stimulating the phospholipase D activity, the compounds of the invention amplify the hormonal effect of abscisic acid to the extent that the activation of the enzyme leads to the production of phosphatidic acid (which mimics the effects of abscisic acid). In this way, they can reveal an antagonism against the gibberellins, ethylene or jasmonates (Grill E., Himmelbach A. (1998) Current Opinion in Plant Biology, 1, 1, 5, 412-418; Ritchie S, Gilroy S (1998) Plant Biology, 95, 5, 3, 2697-2702; Moons A, Prinsen E, Bauw G, Van Montagu M (1997) Plant Cell 9 12, 2243-59).

At present, apart from chemical fertilizers, the control of vegetable development is based chiefly on:
the use of agricultural compositions enriched with trace elements, nitrate, phosphate and potassium compounds, polyamines or certain hormones,
the use of natural or genetically modified micro-organisms, which improve the quality of the soil, promoting vegetable growth or increasing crop yield; these are in particular the Rhizobiacea such as R. meliloti and B. japonicum, free-nitrogen-fixing bacteria, such as Bacillus and Pseudomonas, and fungi such as Penicillium,
the development of transgenic plants. This technology has come up against legal problems and strong opposition on the part of consumers; moreover, it has not yet resulted in satisfactory uses in the biofertilizer sector.

One of the aims of the present invention is to provide new compositions which can be used in the phytosanitary field and in biofertilization, and more particularly to combat abiotic stress in plants, and to control flowering and fructification.

A subject of the present invention is the use of compounds comprising:
one or two X chains, namely an α-D-xylopyranosyl(1,6)-β-D-glucopyranosyl or α-D-xylopyranosyl(1,6)-D-glucopyranose, or β-D-xylopyranosyl(1,4)-β-D-glucopyranosyl or β-D-xylopyranosyl(1,4)-D-glucopyranose chain, or a reduced form of X, also called Xol,
one or two F chains, namely an α-L-fucopyranosyl(1,2)-β-D-galactopyranosyl (1,2)-α-D-xylopyranosyl(1,6)-β-D-glucopyranosyl or α-L-fucopyranosyl(1,2)-β-D-galactopyranosyl (1,2)-α-D-xylopyranosyl(1,6)-β-D-glucopyranose chain, or an α-L-fucopyranosyl (1,2)-β-D-galactopyranosyl(1,2)-β-D-xylopyranosyl(1,4)-β-D-glucopyranosyl or α-L-fucopyranosyl(1,2)-β-D-galactopyranosyl(1,2)-β-D-xylopyranosyl (1,4)-D-glucopyranose chain, or a reduced form of F, also called Fol,
and at least one G chain, namely a β-D-glucopyranosyl or D-glucopyranose unit, substituted or not substituted in position 4, or areduced form of G, also called Gol,
said X, F, and G chains being linked to each other in a random order, and comprising, if appropriate, the following modifications: (i) modification of hydroxyl groups, namely acetylated or methoxylated or acylated derivatives, whose glucose residue at the terminal position is reduced or not, (ii) modification of the terminal reducing unit, such as by reducing amination, (iii) oxidation, in position 6 of the accessible Gal and Glc residues,
   said compounds having the property of:
stimulating glutathione reductase,
and/or of stimulating-phospholipase D in plants,
and/or of stimulating glycosylhydrolases,
   within the scope of uses linked to the above-mentioned properties of said compounds, namely:
the adaptation of plants to an abiotic stress, such as adaptation to the cold, or to a hydric stress such as drought, humidity or salinity,
the control of flowering,
the control of fructification,
the induction of defence reactions against pathogens such as bacteria, viruses, fungi.
   with the exclusion of the above-mentioned use of the compound of formula XXFG.

By control of flowering is meant more particularly control of the key phases of the flowering process such as antheresis (Wang M, Hoekstra S, van Bergen S, Lamers G E, Oppedijk B J, Heijden M W, de Priester W, Schilperoort R A (1999) Plant Mol Biol 39, 3: 489-501), or the development of flower buds (Lim C O, Lee S I, Chung W S, Park S H, Hwang I, Cho M J (1996), Plant Mol Biol, 30, 2, 373-379), such as the floral induction or abscission phases (Colasanti J, Sundaresan V (2000) Trends Biochem Sci, 25, 5, 236-240.

By control of fructification is meant more-particularly control of the triggering and/or duration of the maturation phase (Fan L, Zheng S, Wang X (1997) Plant Cell, 9, 12, 2183-9; Ryan S N, Laing W A, Mc Canus M T (1998), Phytochemistry, 49, 4, 957-963), control of cell wall metabolism with respect to the accumulation of sugars and phenols (Fillion L, Ageorges A, Picaud S, Coutos-Thevenot P, Lemoine R, Romieu C, Delrot S (1999) Plant Physiol 120 (4): 1083-94), and control of leaf and fruit abscission (Gomez-Cadenas A, Mehouachi J, Tadeo F R, Primo-Millo E, Talon M (2000), Planta, 210, 4, 636-643).

The induction of defence reactions against pathogens is, with respect to the elicitation of PR-proteins, in particular of the enzymes 1,3-β D glucanase and endochitinase, also known to be involved in plant development (Munch-Garthoff S, Neuhaus J M, Boller T, Kemmerling B, Kogel K H (1997) Planta 201, 2, 235-44; Buchter R, Stromberg A, Schmelzer E, Kombrink E (1997) Plant Mol Biol 35, 6, 749-61; Robinson S P, Jacobs A K, Dry I B (1997) Plant Physiol 114, 3, 771-8).

The control of metabolic and catabolic modifications of which certain tissues are the object in differentiation or senescence periods, is in accordance with the elicitation of the enzymes 1,4-βD-glucanase and β-D-xylosidase (Trainotti L, Spolaore S, Ferrarese L, Casadoro G (1997) Plant Mol Biol 34 (5): 791-802; Kalaitzis P, Hong S B, Solomos T, Tucker M L (1999) Plant Cell Physiol 40(8), 905-8).

A more particular subject of the invention is the above-mentioned use of compounds defined above, corresponding to acetylated derivatives chosen from:
the mono-acetylated forms in position 2 or 3 or 4 for xylose, or in position 3 or 4 or 6 for galactose, or in position 2 or 3 or 4 or 6 for glucose, or in position 2 or 3 or 4 for fucose,
the di-acetylated forms in position 2 and 3, 2 and 4, 3 and 4, 2 and 6, 3 and 6, or 4 and 6 for glucose, or in position 2 and 3, 2 and 4, or 3 and 4 for xylose, or in position 3 and 4, 3 and 6, or 4 and 6 for galactose, or in position 2 and 3, 2 and 4, or 3 and 4 for fucose, or any combination taking into account two monoacetylated sugars making up the molecule,
the tri-acetylated forms in position 2, 3 and 4 for xylose, or in position 2, 3 and 4, or 2, 3, and 6 for glucose, or in position 3, 4, and 6 for galactose, or in position 2, 3, and 4 for fucose, or any combination taking into account three mono-acetylated sugars or a mono-acetylated sugar and a di-acetylated sugar making up the molecule,
the tetra-acetylated to totally acetylated forms, or any combinations of the different sugars, acetylated or not, making up the molecule.

A more particular subject of the invention is the above-mentioned use of compounds in which the sugars are (L) or (D) glycosyl residues, optionally in reduced form, and/or in α or β form, if appropriate, in pyranose or furanose form, and are interconnected by bonds of the 1→2, 1→3, 1→4, or 1→6 type, and more particularly of the α1→2 type in the case of the bond of a fucose to a galactose, β1→2 in the case of the bond of a galactose to a xylose, β1→4, in the case of the bond of a glucose to a glucose, or α1→6, in the case of the bond of a xylose to a glucose.

A yet more particular subject of the invention is the above-mentioned use of compounds comprising an osidic structure chosen from those of the following formulae:
(X)ₐ(F)_{b}(G)_{c}
(X)ₐ(G)_{c}(F)_{b}
(F)_{b}(X)ₐ(G)_{c}
(F)_{b}(G)_{c}(X)ₐ
(G)_{c}(X)ₐ(F)_{b}
(G)_{c}(X)ₐ(F)_{b}
in which:
G, X and F are as defined above,
a, b, and c, independently of each other represent 1, or 2.

A more particularly subject of the invention is the above-mentioned use:
of compounds comprising an osidic structure of the following formula XFG: 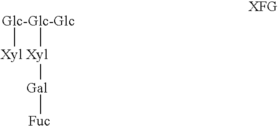
or of compounds comprising a structure derived from XFG corresponding to the following formulae XGF, FXG, FGX, GFX, and GXF: 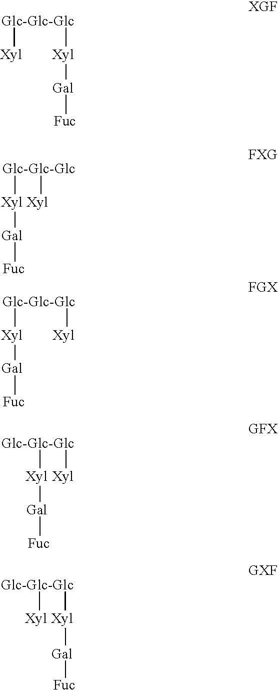
the glucose residue at the terminal position of said compounds being reduced or not, or comprising structures derived by modification as defined above.

The invention also relates to the above-mentioned use, of compounds chosen from the following: XFXG, XFGX, FGXX, FXGX, FXXG, GXXF, GXFX, GFXX, XXGF, XGXF, XGFX.

The invention relates more particularly to the above-mentioned use, of the compound of formula

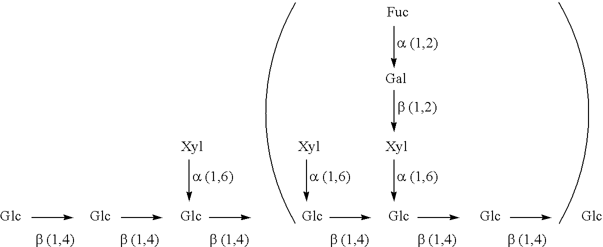

The invention relates more particularly also to the above-mentioned use, of compound XFG of formula

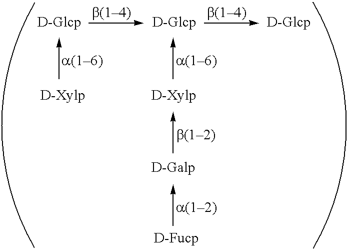

A subject of the invention is also the above-mentioned use of polymers or oligomers comprising as monomeric unit, compounds as defined above, said polymers or oligomers comprising between 2 and approximately 300 monomeric units, in particular between 2 and approximately 100 units, or between 2 and approximately 50 units, or between 2 and approximately 20 units, in particular between 5 and 12 units.

A more particular subject of the invention is the above-mentioned use of above-mentioned polymers comprising a number of monomeric units defined above less than or equal to 12, and preferably less than or equal to 5 (namely polymers whose degree of polymerization DP is less than or equal to 12, and preferably less than or equal to 5).

A subject of the invention is also the above-mentioned use of successive chains of at least two monomeric units defined above, at least one of the units of said chains being different to the other unit or units.

A more particular subject of the invention is the above-mentioned use of chains of units as defined above, in which the number of units is less than or equal to 12, preferably less than or equal to 5.

A subject of the invention is also a process for the stimulation of glutathione reductase in plants, characterized in that it comprises a stage of plant treatment with at least one compound defined above, in particular by irrigation of the soil in which these plants are cultivated, with a composition comprising said compound, or by coating the seeds with such a composition, or by foliar spraying of such a composition in the field on the plants to be treated.

A subject of the invention is also the use of a process for the stimulation of the above-mentioned glutathione reductase, for the implementation of a process for adaptation of the plants to an abiotic stress, such as adaptation to the cold, or to a hydric stress such as drought, humidity or salinity.

The invention also relates to a process for the stimulation of phospholipase D production in plants, characterized in that it comprises a stage of plant treatment with at least one compound defined above, in particular by irrigation of the soil in which these plants are cultivated, with a composition comprising said compound, or by coating the seeds with such a composition, or by foliar spraying of such a composition in the field on the plants to be treated.

A more particular subject of the invention is the use of the above-mentioned process for the stimulation of phospholipase D production, for the implementation of a process for the control of flowering, and more particularly a process for the control of floral induction, of flowering duration, and of flower abscission, and/or for the implementation of a process for the control of plant fructification, and more particularly of a process for the control of the triggering and duration of fruit maturation, of leaf and fruit abscission.

A subject of the invention is also a process for the stimulation of the production of glycosylhydrolases in plants, characterized in that it comprises a stage of treatment of the plants with at least one compound as defined above, in particular by irrigation of the soil in which these plants are cultivated, with a composition comprising said compound, or by coating the seeds with such a composition, or by foliar spraying of such a composition in the field on the plants to be treated.

The invention more particularly relates to the use of the above-mentioned process for the stimulation of the production of glycosylhydrolases, for the implementation of a process for the induction of defence reactions against pathogens, such as bacteria, viruses, fungi, and/or control of certain plant development phases (germination, fertilization, cell differentiation during flowering or fructification).

Advantageously, the above-mentioned compositions comprising at least one compound defined above and used within the scope of the present invention, are presented as agricultural inputs in solid form (in particular powder, granules, pellets), or in liquid form (in particular in aqueous solution), combined or not combined with other agricultural input compounds.

Of the plants capable of being treated-within the scope of the present invention, agronomically useful plants, such as the vine, fruit trees (in particular apple, pear, walnut), cereals (in particular rice, barley), oleaginaceous plants (in particular soya, rape, sunflower), protein plants (in particular peas), and market garden crops (in particular tomatoes) can chiefly be mentioned.

The invention also relates to the above-mentioned use of compounds defined above, such as obtained:
from plants, in particular by extraction of seeds, leaves, roots, fruit, in particular from apples (Malus malus L., Rosaceae), in particular according to the method described in Vincken J P, Beldman G, Niessen W M A, Voragen A G J (1996) Carbohydrate Polymers, 29, 1, 75-85; Spronk B A, Rademaker G J, Haverkamp J, Thomas-Oates J E, Vincken J P, Voragen A G, Kamerling J P, Vliegenthart J F (1997) Carbohydrate Research, 305, 2, 233-242); the process takes place in 3 stages: a) extraction of the xyloglucan polymer of the biomass by alkaline treatment followed by a depectinization by enzymatic route; b) hydrolysis of the polymer using cellulases and more particularly endo 1-4 β D glucanase isolated from Trichoderma viride; c) purification of oligomers by gel-permeation chromatography on Bio-Gel P-2 followed by an anion-exchange chromatography on a Dionex system.
from plant cell suspensions of, in particular
Rubus fruticosus L. in particular according to Joseleau J P, Cartier N, Chambat G, Faik A, K Ruel (1992), Biochimie, 74, 81-88;
Rosa sp. in particular according to Fry S C (1989) J. Exp. Bot. 40, 1-11; Mc Dougall, G. J. Fry S C (1991) Carbohydrate Research 219, 123-132,
the process takes place in 3 stages: a) extraction of the xyloglucan polymer of the cell walls by alkaline treatment coupled with a depectinisation by chemical route or by deproteinization coupled with an alkaline treatment; b) hydrolysis of the polymer using cellulases and more particularly endo 1-4 β D glucanase isolated from Trichoderma viride; c)-purification of oligomers by gel-permeation chromatography on Bio-Gel P-2 followed by a fractionation by anion-exchange chromatography on a Dionex system or by gel-permeation chromatography on Bio-Gel P-2 followed by a fractionation by reversed-phase chromatography on a C₁₈ column or by anion-exchange on a Dionex system; the cellulases can be enzymes obtained by fermentation of bacterial strains which have been genetically modified or not or obtained by recombinant route.

A subject of the invention is also the above-mentioned use of compounds defined above, as obtained:
by chemical synthesis, in particular according to the method described in Pavlova Z N, Ash A O, Vnuchkova V A, Babakov A V, Torgov V I, Nechaev O A, Usov A I, Shibaev V N (1992) Plant Science 85, 131-134; or based on the work of Watt D. K., Brasch D. J, Larsen D. S, Melton L. D, Simpson J Carbohydrate Research, 325, 2000, 300-312,
by chemoenzymatic synthesis, in particular from xyloglucan oligomers modified by the activity of endo-transglycosylase (β-D-glucosidase, α-(β) L-xylosidase, β-D-galactosidase) according to York W. S., Harvey L. K., Guillen R., Alberheim P., Darvill A., Carbohydrate Research, 1993, 248, 285-301 or by the activity of endo transxyloglucanases according to G. Maclachlan, C. Brady, Plant Physiology, 105, 1994, 965-974) or by the activity of fucosyltranferase(s) of vegetable or animal origin according to Baydoun E. A.-H., Abdel-Massih R. m, Dani D, Rizk S, Bret C. T, Journal of Plant Physiology, 158, 2000, 145-150; Faik A, Bar Peled M, DeRocher A E, Zeng W, Perinn R M, Wilkerson C, Raikhel N V, Keegstra K J Biol Chem, 2000, 275, 20,
by recombinant route,
by hydrolysis of the xyloglucan polymer or from oligomers as obtained from polymers or from glycans representing the glycan part of glycoproteins from enzymatic degradation of xyloglucans present in biomasses such as:
1/ fruits and vegetables, and more particularly, peppers, tomatoes, potatoes, olives and apples, or residues produced by these biomasses (<<pomaces >> etc.) according to the methods described in:
Spronk B. A., Rademaker G. J., Haverkamp J., Thomas-Oates J. E., Vincken J. P., Voragen A. G., Kamerling J. P., Vliegenthart J. F., Carbohydrate Research, 305, 1997, 233-242,
Renard C. M. G. C., Lomax J. A., Boon J. J., Carbohydrate Research, 232, 1992, 303-320,
Vincken J. P., Beldman G., Niessen W. M. A., Voragen A. G. J., Carbohydrate Polymers, 29 (1996) 75-85;
2/ cell suspensions (Rubus fructicosos, sycamore) according to:
Joseleau J. P., Cartier N., Chambat G., Faik, A., Ruel K., Biochimie, 74 (1992) 81-88,
Augur C., Yu L., Ogawa T., Sinay P., Darvill A. G., Albersheim P., Plant Physiology, 99 (1992) 180-185;
3/ by fermentation of microorganisms (bacteria, fungi etc.) genetically modified or not;
4/ seeds (nasturtium, Hymenaea, etc.) according to McDougall G. J., ry S. C., Plant Physiology, 89 (1989) 883-887,
5/ leaves, and more particularly those of Hymenaea courbaril, Busato A. P., Vargas-Rechia C. G., Reicher F., Phytochemistry, 58 (2001) 525-531,
by using enzymes which are:
   xyloglucanases (Garcia-Garrido J. M., Rejon-Palomares A., Ocampo J. A., Garcia-Romera I., Mycol. Res., 103 (1999) 882-886),
   cellulases, and more particularly endo 1,4 glucanases of Trichoderma viride (Vincken J P., de Keiser A., Belidman G., Voragen A. G., J., Plant Physiology, 108, 1995, 1579-1585), of Tricoderma. Reesei (Hasper A. A., Dekkers E., van Mil M., van de Vondervoort P. J. L., de Graaff L. H., Applied and Environmental Microbiology, 68, 2002, 1556-1560) or of Aspergillus oryzae (Kato Y, Matsuda K., Agric. Biol. Chem., 44, 1980, 1759-1766),
   exo cellulases such as those of Irpex lacteus,
said enzymes being:
   either isolated from bacterial or fungal strains genetically modified or not, or obtained by recombinant route (Pauly M, Andersen L N, Kaupinnen S, Kofod L V, York W S, Albersheim P, Darvill A (Glycobiology 1999, 9, 1, 93-100),
   used alone or in mixture preferably combined with pectolytic enzymes (polygalacturonases, pectine esterases, pectine lyases) according to Renard C. M. G. C., Searle can Leewen M. J. F., Voragen A. G. J., Thibault J. F., Pilnik W., Carbohydrate Polymers, 14, 1991, 295-314, or replaced by broad spectrum enzymes of pectolytic type.

The mixture of oligomers obtained by hydrolysis of the polymer is fractionated according to degree of-polymerization (DP) by steric gel-permeation chromatography on Bio-Gel column and the DP oligosaccharides which are of interest are then separated according to structural characteristics by high performance liquid chromatography (HPLC).

Several systems can be used from anion-exchanger chromatography (HPAEC) or chromatography on a DIONEX column (Vincken J. P., Beldman G., Niessen W. M. A., Voragen A. G. J., Carbohydrate Polymers, 29, 1996, 1, 75-85), and normal phase (Kakegawa K., Edashige Y., Ishii T., Phytochemistry, 47, 1998, 767-771) or reversed-phase affinity chromatography (Waft D. K., Brash D. J., Larsen D. S., Melton L. D., Carbohydrate Polymers, 39, 1999, 165-180).

The structure of each oligosaccharide is then confirmed by spectroscopic methods (NMRoand mass spectrometry) (York W. S., van Halbeek H., Darvill A. G., Albersheim P., Carbohydrate Research, 200, 1990, 9-31).

The invention is further illustrated by the detailed description which follows of the elicitor potential of compounds according to the invention in the vine, namely the effect of inducing cold-resistance.

Plants originating from different vine varieties, including Pinot noir, are used for the study. Each sample, comprising 5 plants, is treated with foliar spraying at different vegetative stages on the BBCH scale with the xyloglucan elicitor: heptasaccharide XFG in solution at variable doses; the spraying of 2.5 ml of solution per plant is carried out using a hand sprayer (deviation of +/−1%).

After use of the elicitor, the plants are exposed to cold stress of variable duration and intensity. After exposure to the cold, the plants are placed in a climatic chamber at 20° C. with a 12-hour day/night alternation. The appearance of the leaves is observed 24 hours, 72 hours after removal from the cold. The effects of the cold are evaluated by observing the foliar necroses induced by frost and the plants are kept for several months in order to monitor their subsequent development.

The results on 50 plants and relating to 10 repetitions are expressed by the protection index 1_{f}(%)=100-P; P, being the proportion of foliar necroses. The results relating to the control plants C treated with water and the plants elicited by heptasaccharide: XFG or XFGol are expressed by the protection index 1_{f}(%) measured 24 hours after the stress.

Results:
Vine variety: Pinot noir at BBCH 13 stage

Temperature: 3.5° C. duration 180 min

| | C (1_{f}) | XFG (1_{f}) | XFGol (1_{f}) |
|---|---|---|---|
| dose: 33 mg/l | 75 | 100 | 100 |
| dose: 33 μg/l | 75 | 100 | 100 |

Moreover, it is noted that the use of the elicitor:
also provides frost-protection for the vine varieties Chenin, Chardonnay, Cabernet sauvignon, Pinot meunier, Merlot, Grolleau,
does not change the development of the vine,
has a frost-protection effect which lasts for several days.

The plants treated by the xyloglucan elicitor at a dose of 3.3 mg/l resist cold stress which destroys the leaves of the controls treated with water: the colouring of the leaves of the elicited plants remains normal instead of changing to dark green when thawing (as is observed for the controls treated with water), and no sign of necrosis appears after 24 hours as is observed for the controls treated with water).

It is noted that the use of the elicitor does not cause any interference in the development of the plant given that the development of the elicited plants after the cold stress is comparable to that of the control plants not exposed to the cold.

1-15. (canceled)

16. A compound comprising:
one or two X chains, namely an α-D-xylopyranosyl(1,6)-β-D-glucopyranosyl or α-D-xylopyranosyl(1,6)-D-glucopyranose, or β-D-xylopyranosyl (1,4)-β-D-glucopyranosyl or β-D-xylopyranosyl(1,4)-D-glucopyranose chain, or a reduced form of X, also called Xol,
one or two F chains, namely an α-L-fucopyranosyl(1,2)-β-D-galactopyranosyl (1,2)-α-D-xylopyranosyl(1,6)-β-D-glucopyranosyl or α-L-fucopyranosyl (1,2)-β-D-galactopyranosyl(1,2)-α-D-xylopyranosyl(1,6)-D-glucopyranose chain, or an α-L-fucopyranosyl(1,2)-β-D-galactopyranosyl(1,2)-β-D-xylopyranosyl (1,4)-β-D-glucopyranosyl or α-L-fucopyranosyl(1,2)-β-D-galactopyranosyl (1,2)-β-D-Xylopyranosyl(1,4)-D-glucopyranose chain, or a reduced form of F, also called Fol,
and at least one G chain, namely a β-D-glucopyranosyl or D-glucopyranose unit, substituted or not substituted in position 4, or a reduced form of G, also called Gol,
said X, F, and G chains being linked to each other in a random order, and comprising, if appropriate, the following modifications: (i) modification of hydroxyl groups, namely acetylated or methoxylated or acylated derivatives, whose glucose residue at the terminal position is reduced or not, (ii) modification of the terminal reducing unit, such as by reducing amination, (iii) oxidation, in position 6 of the accessible Gal and Glc residues,
said compounds having the property of:
stimulating glutathione reductase,
and/or of stimulating phospholipase D in plants,
and/or of stimulating glycosylhydrolases,
within the scope of uses linked to the above-mentioned properties of said compounds, namely:
the adaptation of plants to an abiotic stress, such as adaptation to the cold, or to a hydric stress such as drought, humidity or salinity,
the control of flowering,
the control of fructification,
the induction of defence reactions against pathogens such as bacteria, viruses, fungi
with the exclusion of the above-mentioned use of the compound of formula XXFG.

17. The compound according to claim 16, corresponding to acetylated derivatives chosen from:
the mono-acetylated forms in position 2 or 3 or 4 for xylose, or in position 3 or 4 or 6 for galactose, or in position 2 or 3 or 4 or 6 for glucose, or in position 2 or 3 or 4 for fucose,
the di-acetylated forms in position 2 and 3, 2 and 4, 3 and 4, 2 and 6, 3 and 6, or 4 and 6 for glucose, or in position 2 and 3, 2 and 4, or 3 and 4 for xylose, or in position 3 and 4, 3 and 6, or 4 and 6 for galactose, or in position 2 and 3, 2 and 4, or 3 and 4 for fucose, or any combination taking into account two monoacetylated sugars making up the molecule,
the tri-acetylated forms in position 2, 3 and 4 for xylose, or in position 2, 3 and 4, or 2, 3, and 6 for glucose, or in position 3, 4, and 6 for galactose, or in position 2, 3, and 4 for fucose, or any combination taking into account three mono-acetylated sugars or a mono-acetylated sugar and a di-acetylated sugar making up the molecule,
the tetra-acetylated to totally acetylated forms, or any combinations of the different sugars, acetylated or not, making up the molecule.

18. The compound according to claim 16 in which the sugars are in α or β form, if appropriate, in pyranose or furanose form, and are interconnected by bonds of the 1→2, 1→3, 1→4, or 1→6 type.

19. The compound according to claim 16 comprising an osidic structure chosen from those of the following formulae:
(X)ₐ(F)_{b}(G)_{c}
(X)ₐ(G)_{c}(F)_{b}
(F)_{b}(X)ₐ(G)_{c}
(F)_{b}(G)_{c}(X)ₐ
(G)_{c}(X)ₐ(F)_{b}
(G)_{c}(X)ₐ(F)_{b}
in which:
a, b, and c, independently of each other represent 1, or 2.

20. The compound according to claim 16 comprising an osidic structure of formula XFG or comprising a structure derived from XFG corresponding to the formulae XGF, FXG, FGX, GFX, and GXF, whose glucose residue at the terminal position is reduced or not, or comprising structures derived by modification.

21. The compound according to claim 16 chosen from the following: XGXG, XFGX, FGXX, FXGX, FXXG, GXXF, GXFX, GFXX, XXGF, XGXF, XGFX.

22. The compound according to claim 16, wherein the compound is of formula

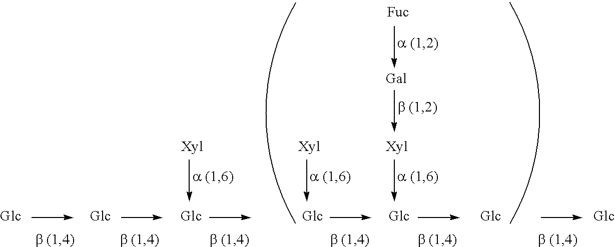

23. The compound according to claim 16, of the XFG compound of formula

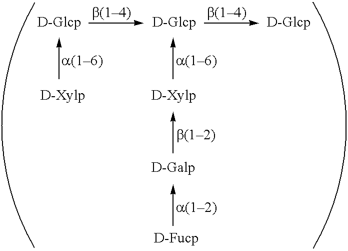

24. The compound according to claim 16, of polymers or oligomers comprising as monomeric unit, compounds, said polymers or oligomers comprising between 2 and approximately 300 monomeric units.

25. A process of stimulating glutathione reductase in plants, characterized in that said process comprises a stage of treating of the plants with at least one compound defined in claim 16, by irrigation of the soil in which these plants are cultivated, with a composition comprising said compound, or by coating the seeds with such a composition, or by foliar spraying of such a composition in the field on the plants to be treated.

26. The process according to claim 25, comprising adaptation of the plants to an abiotic stress, such as adaptation to the cold, or to a hydric stress such as drought, humidity or salinity.

27. Process of stimulation of phospholipase D production in plants, characterized in that it comprises a stage of treatment of the plants with at least one compound according to claim 16 by irrigation of the soil in which these plants are cultivated, with a composition comprising said compound, or by coating the seeds with such a composition, or by foliar spraying of such a composition in the field on the plants to be treated.

28. The process according to claim 27, comprising the control of flowering, and more particularly a process for the control of floral induction, of flowering duration, and of flower abscission, and/or for the implementation of a process for the control of plant fructification, and more particularly of a process for the control of the triggering and duration of fruit maturation, of leaf and fruit abscission.

29. Process for the stimulation of the production of glycosylhydrolases in plants, characterized in that said process comprises a stage of treatment of the plants with at least one compound according to claim 16 by irrigation of the soil in which these plants are cultivated, with a composition comprising said compound, or by coating the seeds with such a composition, or by foliar spraying of such a composition in the field on the plants to be treated.

30. The process according to claim 29, comprising induction of defence reactions against pathogens such as bacteria, viruses, fingi.

31. A method for stimulating glutathione reductase in plants, stimulating phospholipase D production in plants, controlling flowering in plants, controlling plant fructification in plant, controlling fruit maturation in plants, inducing defense reaction of plants, or stimulating production of glycosylhydrolases, comprising administering to a plant in need thereof an effective amount of a compound according to claim 16.
